(19) United States
(12) Patent Application Publication
(10) Pub. No.: US 2026/0047801 A1
(43) Pub. Date: Feb. 19, 2026

(54) DEVICE HAVING A WOUND DRESSING, IMAGING MEANS AND TREATMENT ELEMENT

(71) Applicant: PAUL HARTMANN AG, Heidenheim (DE)

(72) Inventors: Martin Deibler, Herbrechtingen (DE); Pierre Croizat, Herbrechtingen (DE)

(21) Appl. No.: 18/719,021

(22) PCT Filed: Dec. 22, 2022
(86) PCT No.: PCT/EP2022/087395

§ 371 Date: Oct. 16, 2025

(30) Foreign Application Priority Data
Dec. 30, 2021 (EP) 21218300.8

(51) Int. Cl. A61B 5/00

(52) U.S. Cl. CPC A61B 5/4836; A61B 5/0037; A61B 5/0077; A61B 5/445; A61B 5/6833

(57) ABSTRACT

The present invention relates to a device comprising wound dressing and an image recording means. The wound dressing comprises a cover layer, a first transceiver element and a treatment element which has at least two independently activatable regions. The image recording means comprises a digital camera, a first processor, and a second transceiver element configured to receive data from the first transceiver element and transmit data to the first transceiver element. By means of the at least two independently activatable regions, it is possible either, within the wound to be treated, to exert a therapeutic effect only on a portion of the wound to be treated or, within a wound site comprising the wound to be treated and the skin surrounding the wound, to exert a therapeutic effect only on the wound.

The present invention relates to a device comprising a wound dressing, which can exert a therapeutic effect on the wound to be treated, and to an image recording means.

The disadvantage of wound coverings, especially self-adhesive wound dressings with releasable active ingredients or with physical treatment elements such as a thermoelement, for example, is that on account of the fixed form of the wound dressing, the active ingredient or the physical treatment also acts intact regions of the skin or on regions of the wound requiring no treatment. Active wound contact layers that can be matched to the shape of the wound are complicated in terms of application and harbor an additional infection risk during the adjustment.

The invention is based on the object of providing a device allowing an improved treatment of a wound. A further object lies in providing a device allowing a resource-sparing treatment of a wound. A further object of the invention lies in providing a device allowing a precise treatment of those regions of the wound in actual need of treatment. In the process, therapeutic action on the intact skin surrounding the wound should be avoided as much as possible.

The objects are achieved by a device as claimed in claim 1 and by a method as claimed in claim 11.

The device according to the claim on the one hand allows, within a wound to be treated, a therapeutic effect to be selectively exerted only on a portion of the wound to be treated. On the other hand, the device according to the claim allows a therapeutic effect to be exerted only on the actual wound within a wound site, the latter comprising the wound to be treated and the skin surrounding the wound, i.e., the intact skin surrounding the wound to be excluded from the therapeutic effect.

The wound dressing contained in the device comprises a cover layer arranged distant from the wound when the wound dressing is used and a first transceiver element capable of transmitting and receiving data. The wound dressing also comprises a treatment element which has at least two independently activatable regions. By means of the at least two independently activatable regions, it is possible either, within the wound to be treated, to exert a therapeutic effect only on a portion of the wound to be treated or, within a wound site comprising the wound to be treated and the skin surrounding the wound, to exert a therapeutic effect only on the wound.

The device also comprises an image recording means. The image recording means comprises a digital camera, a first processor, and a second transceiver element configured to receive data from the first transceiver element and transmit data to the first transceiver element.

In a preferred embodiment, the treatment element is in direct contact with the wound bed when the wound dressing is used.

In a different preferred embodiment, the wound dressing comprises a wound contact layer. The wound contact layer is arranged on the side facing the wound when the wound dressing is used. If a wound contact layer is provided, the independently activatable regions are preferably arranged in a plane parallel to the wound contact layer.

Each independently activatable region does not have a common area with its adjacent regions and can be activated on an individual basis. The independently activatable regions comprise electronic components which are applied or embedded in a matrix-like arrangement on a flexible substrate such as silicone, for example.

The electronic components of the independently activatable regions may comprise light guides, photodiodes, photosensors, piezoelectric ultrasonic emitters, and piezoelectric ultrasonic sensors, for example.

Should the activatable regions comprise photodiodes, treatment effects can be achieved by the interaction of the photons with the wound, the wound dressing or with photoactivatable agents. For example, the treatment effects can be based on direct local heating, oxygen radical formation or plasmon resonance phenomena with locally induced release of heat, which are conducive to wound healing. In this case, it is necessary to use photodiodes that emit light matched to the desired treatment.

The use of ultrasonic arrays allows the removal of biofilms or bacterial agglomerations by micro-cavitation.

In a preferred embodiment, the device comprises a second processor, which is a component part of the wound dressing. The second processor can control the independently activatable regions of the treatment element. By preference, the second processor is a microcontroller.

The first transceiver element is situated on the wound dressing. The first transceiver element communicates with a second transceiver element. By preference, wireless communication with a transmitter output power of no more than 1000 mW, preferably of no more than 100 mW and particularly preferably of no more than 10 mW should be used to this end. Particularly preferably, the wireless communication is based here on Bluetooth, Bluetooth ultralow energy or Wi-Fi. Data transmission systems such as Zigbee, Wibree or other methods in the radio frequency range are also conceivable.

The electronic components of the independently activatable regions, of the first transceiver element, and of the optional second processor can be supplied with power by way of a battery, inductive charging or by way of energy harvesting. In energy harvesting, the energy available in the surroundings is converted into electrical energy. The present invention preferably uses electromagnetic radiation as energy source. In this context, radio waves with frequencies from 30 kHz to 300 GHz are particularly preferred. Unlike in the prior art, the radio waves preferably need not or need not exclusively be generated by way of a specifically adapted reader. Instead, the radio waves already present in the surroundings can be used. For example, these radio waves may originate from radio towers, radar installations, Wi-Fi networks or Bluetooth connections. For Wi-Fi networks, radio waves at frequencies of 2.4 GHz and a power up to 100 mW, and at frequencies of 5 GHz and a power up to 1 W are of particular interest. Radio waves based on mobile radio networks/GSM are at the frequencies 850 MHz, 900 MHz, 1800 MHz, and 1900 MHz pursuant to the GSM standards.

The wound dressing communicates with an image recording means which receives data from the wound dressing and transmits data to the wound dressing by means of the second transceiver element. Using the image recording means, it is possible to determine either the actual wound within the wound site or the region to be treated of the wound within a wound. Just like the first transceiver element, the second transceiver element can for example be a Bluetooth transceiver or a Wi-Fi transceiver in this case. In addition to the second transceiver element configured to receive data from a first transceiver element and transmit data to the latter, the image recording means comprises at least one digital camera and at least one first processor. In this case, the camera can be cameras in smartphones, camera systems with CCD, or CMOS sensor systems with a communications interface. The first processor can process the signal received by the first transceiver element and is configured to carry out the following steps:
to segment a first image of the wound recorded by the camera (the first image is recorded when the wound dressing is absent). In this case, segmentation is understood to mean the division of the image into at least two regions according to at least one criterion. By preference, these regions do not overlap,
to correlate a second image, which was recorded by the camera, and the wound and a wound dressing applied to the wound with the first image of the wound and determine the position of the wound dressing relative to the area of the wound (thus, the second image is recorded after the wound dressing has been applied to the wound),
to ascertain those independently activatable regions of the wound dressing located over the area of the wound,
to activate the independently activatable regions of the wound dressing located over the area of the wound. The instruction for activation or selective activation is transmitted from the image
processing means to the wound dressing by means of the second transceiver element and the first transceiver element.

“Correlation” or “correlating” is understood to mean that visual features in a first image are linked to visual features in a second image. In this case, visual features in the first image are uniquely assigned to visual features in the second image. By preference, such visual features are individual points of orientation determined previously in the first image.

By preference, the image recording means comprises a storage unit.

Should the independently activatable regions of the wound dressing comprise sensors such as photosensors or piezoelectric ultrasonic sensors, the data recorded by the sensors can be sent to the second transceiver element of the image recording means. The image recording means comprises an output means in a preferred embodiment. The output means can be an electronic visual display. A graphical representation of the data, which allows conclusions to be drawn about the course of healing of the wound and which can thus serve for the observation of the course of wound healing, can be reproduced on the output means. The graphical representation of the data is calculated by way of the first processor. Further properties of the wound can also be determined and documented with the aid of the first processor. By preference, these properties of the wound comprise the wound dimension, the wound depth, and/or the tissue composition. In another preferred embodiment, the independently activatable regions of the wound dressing comprise in addition treatment elements, which can be realized as light guides, photodiodes, and ultrasonic emitters, in addition to the sensors. The data recorded by the sensors are sent to the first transceiver element of the image recording means. The first processor contained in the image recording means determines the current state of the wound and stores the current state of the wound in the storage unit. If at least one state of the wound was stored in the storage unit, then the current state of the wound is compared with at least one of the preceding states of the wound using the first processor. The activation of the treatment elements of the independently activatable regions of the wound dressing can be adjusted in a manner dependent on the current state of the wound. For example, in this case, the adjustment to the current State of the wound may comprise a reduction in the number of active treatment elements of the independently activatable regions or a change in the frequency should photodiodes be used. The activation of the treatment elements of the independently activatable regions is transmitted to the first transceiver element via the second transceiver element.

The first image of the wound and the immediate surroundings of the wound is recorded by means of the image recording means. Accordingly, the first image must comprise the regions of skin surrounding the wound. The first image of the wound is segmented. For the purpose of segmenting the image of the wound, differences between two parameter values are calculated by preference. The parameter values preferably comprise brightness values. In the case of brightness values, the differences between two parameter values are preferably calculated between the parameter values of a pixel and between the parameter values of its nearest neighbor. For example, a boundary of a segment is assumed if the difference exceeds a limit value. A single-shot detector (SSD) can be an example of an algorithm based on the difference between two parameter values. Pattern recognition algorithms are used during the segmentation in another preferred embodiment. Visual features of objects are used to divide an image into a plurality of segments. Possible algorithms may comprise k-means clustering, thresholding methods or neural networks, in particular convolutional neural networks.

The image recording means is used to record the second image of the wound as soon as the wound dressing was applied. The second image comprises the applied wound dressing, and also regions of the wound if the wound is not covered by the wound dressing. Like in the case of the first image, the second image should also contain the immediate surroundings of the wound in the recording. By preference, an image excerpt that is as similar as possible is sought after for the first and the second image. Ideally, the second image is recorded from the same distance from the wound and at the same angle with respect to the wound as the recording of the first image. The first image is correlated with the second image in order to determine the position of the wound dressing relative to the wound. In a preferred embodiment, the first and the second image are correlated by means of orientation points. For example, the orientation points can be nevi, outlines of the body part with the wound, scars, skin defects, hairs, markings applied to the intact skin, or markers placed next to the wound. At least three of the three or more orientation points must be visible both in the first image and in the second image. Non-symmetrical orientation points visible both in full in the first image and in the second image are preferred.

The position of the orientation points relative to one another is determined in the first and second image. The position of the orientation points relative to one another can be determined by way of the distance between the orientation points or a combination of angles between the connecting lines of the orientation points and the distance between the orientation points. The change in the position of the orientation points relative to one another provides information about the recording angle and/or the distance at which the first image was recorded relative to the recording of the second image. The distortion of the orientation points themselves can also provide information about the recording angle or the distance. The position of the wound dressing can be determined relative to the orientation points. In another preferred embodiment, autocorrelation-based algorithms are used to determine the position of the wound dressing on the patient on the basis of the first image and the second image.

By preference, at least one visible label is applied to the cover layer. The visible label can facilitate the ascertainment of the accurate position Of the wound dressing since the geometry and the size of the visible label are known. A distortion of a known geometry with a defined size and at least one symmetry-breaking property can allow conclusions to be drawn about a dimension or a recording angle. The visible label consists of at least three points. The visible label can likewise comprise patterns, writing, logos, and/or pictures.

The position of the wound dressing relative to the orientation points and hence the position of the wound dressing relative to the position of the segmented wound image are determined. The absolute size of the objects, in particular the wound, depicted in the first and second image can be ascertained by way of the visible labels on the wound dressing.

The independently activatable regions located over the wound are assigned to a region of the wound to be treated.

By preference, only those independently activatable regions of the wound dressing located over the wound in full are activated.

In another preferred embodiment, an independently activatable region is only activated if specified condition is met. The specified condition can be the enablement by a user or by a third person. In an alternative or in addition, the specified condition can be one or more values of one or more parameters characterizing the property of the wound. The parameter can comprise a read value. The parameter may also comprise a positive identification (i.e., a successful recognition) of the tissue type or a wound condition in the area.

The independently activatable regions of the wound dressing located over the area of the wound are activated. Thus, the treatment element or the treatment elements can be activated selectively, with the therapeutic effect remaining restricted to the actual wound area. Use of the present invention also makes it possible to selectively treat selected regions within the wound.

### DRAWINGS

For the better understanding of the present invention and its advantages, reference is now made to the following embodiments in conjunction with the associated drawings. The invention is explained in detail below on the basis of exemplary embodiments which are specified in the schematic drawings.

FIG. 1

A schematic illustration of an embodiment of a wound dressing proposed by the present patent application, which allows the user to selectively exert a therapeutic effect only on a portion of a wound to be treated within the wound to be treated or to exert a therapeutic effect only on the actual wound within a wound site. A section through the wound dressing is depicted.

FIG. 2

A schematic illustration of an embodiment of the treatment element in plan view.

FIG. 3

A schematic illustration of a method for selectively treating a wound region.

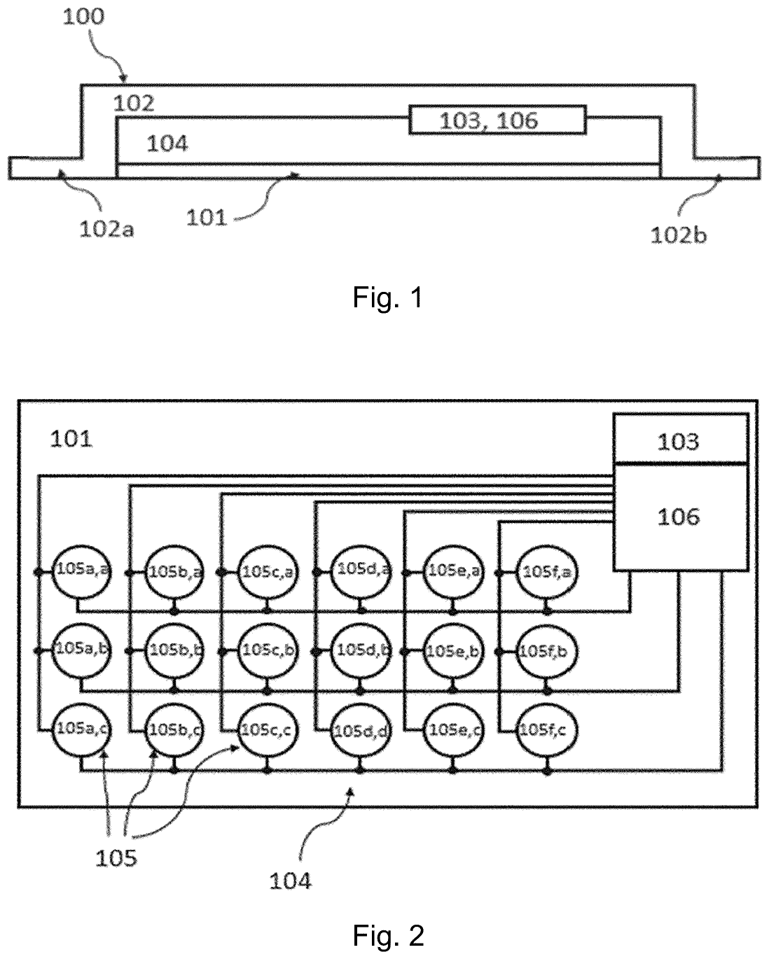

FIG. 1 shows a preferred embodiment of the wound dressing 100. The wound dressing 100 comprises a wound contact layer 101, a cover layer 102, and a treatment element 104, with the treatment element 102 being arranged between the wound contact layer 101 and the cover layer 102. The wound contact layer 101 lies directly on the wound site or the wound of the patient when the wound dressing 100 is used. The cover layer 102 may comprise adhesive regions 102a, 102b on the side of the cover layer that faces the patient. The adhesive regions 102a, 102b serve to secure the wound dressing 100.

Moreover, the wound dressing 100 comprises a first transceiver element 103 and a power source 106. The power source 106 supplies the treatment element 104 and the first transceiver element 103 with power. The power source 106 can be a battery. Alternatively, the power source 106 can provide power by means of energy harvesting.

FIG. 2 shows a possible embodiment of the treatment element 104 of the wound dressing 100. The treatment element comprises independently activatable regions. The independently activatable regions 105 are preferably arranged in a plane parallel to the wound contact layer 101.

Each independently activatable region 105 does not have a common area with its adjacent regions and also can be activated on an individual basis. The independently activatable regions 105 comprise electronic components which are applied or embedded in a matrix-like arrangement on a flexible substrate such as silicone, for example. The electronic components of the independently activatable regions may be light guides, photodiodes, photosensors, piezoelectric ultrasonic emitters, and piezoelectric ultrasonic sensors.

The electronic components 105a,a to 105f,c are connected such that they can be activated on an individual basis.

The electronic components 105a,a to 105f,c are supplied with power by means of the power source 106. The first transceiver element 103 is also coupled to the power source 106. Data relating to the state of the wound are transmitted to a second transceiver element (not depicted) by means of the first transceiver element 103. Likewise, the first transceiver element 103 may receive instructions regarding the activation of the independently activatable regions 105 by way of the second transceiver element.

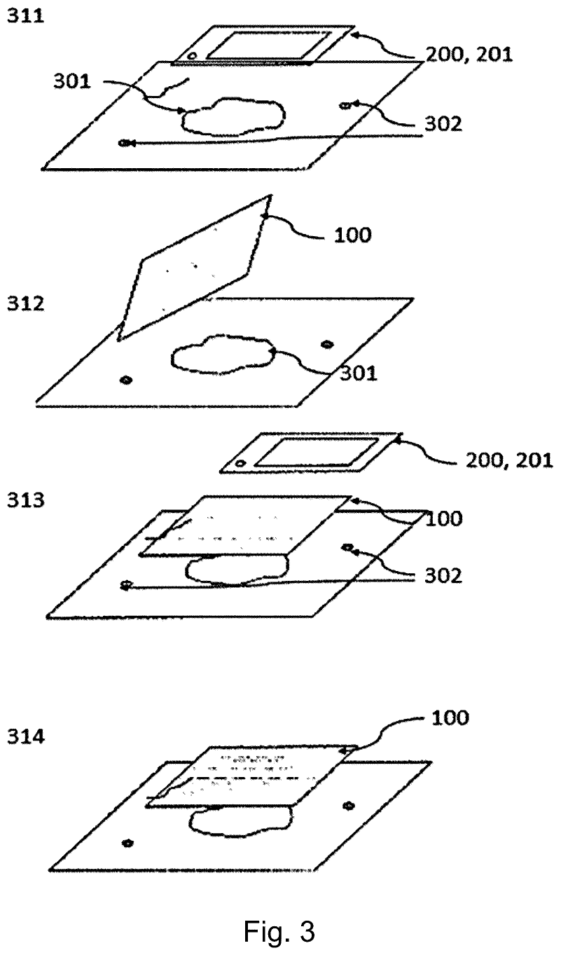

By way of example, FIG. 3 shows step by step (sequence 311-314) how the kit of wound dressing 100 and image recording means 200 can be used:

In step 311, a first image of the wound 301 is recorded by means of the camera 201 in the image recording means 200. The image recording means 200 can be a Bluetooth-capable smartphone with a camera. The first image of the wound 301 is segmented by means of a digital method. The optical orientation points 302 are determined. For example, the orientation points are nevi, applied markers, or outlines of the body part on which the wound is situated.

The wound dressing 100 is applied to the wound 301 in step 312.

A second image of the wound 301 with the wound dressing 100 applied in step 312 is recorded by the camera 201 in step 313. The second image of the wound is correlated with the first image of the wound. The position of the wound dressing 100 relative to the area of the wound is determined with the aid of the orientation points 302.

The independently activatable regions of the wound dressing 100 are correlated with the area of the wound by means of the first image and the second image, and with the aid of the orientation points 302.

Subsequently, additional covers or additional bandaging material can be applied to the wound site.

In step 314, the independently activatable regions of the wound dressing 100 which meet a specified condition are activated. The condition may comprise at least 100% of the independently activatable regions being located over the area of the wound 301.

1. A device comprising a wound dressing (100) and an image recording means (200),
wherein the wound dressing (100) comprises
a. a cover layer (102) arranged distant from the wound when the wound dressing (100) is used,
b. a first transceiver element (103) capable of transmitting and receiving data, and wherein the image recording means (200) comprises
a. a digital camera,
b. a first processor,
c. a second transceiver element configured to receive data from the first transceiver element (103) and transmit data to the first transceiver element (103),
and wherein the wound dressing (100) comprises a treatment element (104) which has at least two independently activatable regions, such that, either within a wound to be treated, a therapeutic effect is exerted only on a portion of the wound to be treated or that, within a wound site comprising the wound to be treated and the skin surrounding the wound, a therapeutic effect is exerted only on the wound.

2. The device of claim 1, wherein the treatment element (104) is in direct contact with a wound bed when the wound dressing (100) is used.

3. The device of claim 1, wherein the wound dressing (100) comprises a wound contact layer (101) arranged on a side facing the wound when the wound dressing (100) is used.

4. The device of claim 3, wherein the treatment element (104) is arranged between the wound contact layer (101) and the cover layer (102).

5. The device of claim 2, wherein the at least two independently activatable regions are arranged in a plane parallel to the wound bed.

6. The device of claim 1, wherein the at least two independently activatable regions each comprise at least one of the components selected from the group consisting of a photodiode, a photosensor, a light guide, a piezoelectric ultrasonic emitter, a piezoelectric ultrasonic sensor, an electrically conductive element, and a thermoelement.

7. The device of claim 1, wherein the device is configured such that the data can be transmitted and received contactlessly by the first transceiver element (103) to the second transceiver element.

8. The device of claim 1, wherein the wound dressing (100) comprises at least one visible label on a side of the cover layer (102) facing away from the wound.

9. The device of claim 1, wherein the wound dressing (100) comprises a second processor.

10. The device of claim 1, wherein the first processor is configured
to segment a first image, which comprises an image of the wound to be treated and which was recorded by the image recording means (200), such that the first image is divided into one or more segments and the one or more segments of the first image which represent the wound to be treated are labeled as area of the wound,
to correlate a second image, which comprises an image of the wound dressing (100) on the wound to be treated and which was recorded by the image recording means (200), with the first image such that the position of the wound dressing (100) is determined relative to the area of the wound,
to ascertain those independently activatable regions of the wound dressing (100) which are located over the area of the wound,
to activate or selectively activate the independently activatable regions of the wound dressing (100) which are located over the area of the wound, and
wherein the instruction for activation or selective activation is transmitted from the image processing means to the wound dressing (100) by means of the second transceiver element and the first transceiver element.

11. A method for controlling the treatment elements of the device of claim 1, comprising the steps:
recording a first image comprising an image of the wound by means of the image recording means,
segmenting the first image such that the first image is divided into one or more segments and the one or more segments of the first image which represent the wound to be treated are labeled as area of the wound,
providing the wound dressing (100) and applying the wound dressing (100) to the wound to be treated,
recording a second image comprising an image of the wound dressing (100) on the wound to be treated by means of the image recording means,
correlating the first image and the second image such that the position of the wound dressing (100) is determined relative to the area of the wound,
transmitting to transmit an instruction from the image recording means (200) to the wound dressing (100) by means of the first transceiver element and the second transceiver element,
ascertaining those independently activatable regions of the wound dressing (100) located over the area of the wound, and
activating or selectively activating the independently activatable regions of the wound dressing (100) located over the area of the wound, wherein the instruction for activation or selective activation of the independently activatable regions of the wound dressing (100) is transmitted from the image processing means to the wound dressing (100) by means of the second transceiver element and the first transceiver element.

12. The method of claim 11, wherein a number of the at least two independently activatable regions of the wound dressing (100) are configured to be activated or selectively activated if at least one additional specified condition is satisfied.

13. The method of claim 12, wherein the specified condition is an enablement by a user or a third person and/or a value of a parameter characterizing the property of the wound.

14. The method as claimed in claim 11, wherein those independently activatable regions of the wound dressing (100) located over the area of the wound are ascertained by means of at least one visible label on a side of the cover layer (102) facing away from the wound.
